(19) United States
(12) Patent Application Publication
(10) Pub. No.: US 2024/0358396 A1
(43) Pub. Date: Oct. 31, 2024

(54) ENDOSCOPIC SNARE NET RETRIEVAL DEVICE

(71) Applicants: Ken Wah Chow, Davis, CA (US); Kenneth William Chow, Davis, CA (US)

(72) Inventors: Ken Wah Chow, Davis, CA (US); Kenneth William Chow, Davis, CA (US)

(21) Appl. No.: 18/645,634

(22) Filed: Apr. 25, 2024

Related U.S. Application Data
(60) Provisional application No. 63/461,996, filed on Apr. 26, 2023.

(51) Int. Cl. A61B 17/3205; A61B 17/221

(52) U.S. Cl. CPC A61B 17/32056; A61B 2017/2212

(57) ABSTRACT

The invention is directed towards an endoscopic snare net retrieval device with a perpendicular-deploying snare loop and a mesh for efficiently capturing and extracting esophageal obstructions or tissue growths, significantly reducing the risk of perforation and procedure time.

### FIELD OF THE INVENTION

The field relates to endoscopic retrieval devices, especially endoscopic snare nets.

### BACKGROUND OF THE INVENTION

Gastroenterologists have been using a net retrieval device to remove foreign bodies from the obstructed esophagus or an internal cavity of a patient. For the past few decades, there has been very little modifications to the original “Roth Net”™. Most recently, rotatable nets have been marketed.

It can be very frustrating when a foreign body, typically a large piece of meat, is tightly lodged in the esophagus due to a stricture, to a decrease in compliance or a decrease in elasticity. A snare with a net is typically used to “slide” past the lodged bolus prior to the deployment of the net. Since the food bolus is typically lodged tightly, there is no gap between the food bolus and the esophageal wall for the snare net to slide past. Blindly sliding a snare net alongside the lodged food bolus significantly increases the risk of esophageal perforation. In other instances, such as removal of polyps from the colon of a patient, the polyp frequently escapes from the capturing instrumentality and falls away into the colon (or other cavity). Especially in cases where the polyp is large, the effort and time expended in retrieving the severed polyp may rival or even exceed the effort and time required to locate and sever the polyp. Overall, the manipulations necessary to remove a severed polyp generally increase the trauma to the patient, the expense of the surgery and the hospitalization time.

Snare nets are known that tie a portion of the net to a cold loop or loops. Examples of known snare nets are found in U.S. Pat. Nos. 8,016,838; 7,618,437; 6,814,739; 5,906,621; 5,759,187; 5,486,182; 5,336,227; 5,201,740; and 5,190,542. However, these known snare nets have a difficult time forming a pocket for capturing a polyp or foreign object or have complex mechanisms or have excessive netting material that may snag or require a larger diameter lumen to pass through. While some of complex systems solve one or more of the problems, added complexity makes them potentially prone to failure and may add substantial cost to a medical procedure.

Thus, there is a long felt need for a snare retrieval device that can slide past the bolus and the esophageal or internal cavity wall with minimal space requirement, then expand to capture and retrieve the bolus, or for that matter, to facilitate the capture and retrieval of severed polyps.

### SUMMARY OF THE INVENTION

An endoscopic snare net retrieval device for capturing and removing bolus, foreign bodies, or undesired growth tissues from an internal cavity of a patient, comprising: a snare loop; a mesh securely coupled to and cover the entirety of the area within the snare loop; a tube having a lumen configured and arranged therein; a control wire or cable that is disposed within a lumen of the tube such that the snare loop contains the mesh thereon is located at the distal end of the tube and is attached to a distal end of the control wire or cable; an external string or suture that has a first end coupled to a distal end of a control wire or cable via the outer wall of a distal end of the tube, and a second end that anchors the snare loop at a point on the snare loop that is outermost away from the distal end of the tube, and a drive member coupled to a proximal end of the control wire or cable and is exposed at a proximal end of the tube such that the actuation of the drive member move the snare loop via the control wire or cable, to fully retrieve the snare loop within the lumen of the tube at the distal end of the tube, or to release and expand the loop, the external string or suture, and the mesh coupled thereon at the distal end of the tube away from the tube, such that the expanded snare loop is about 90° apart from the distal end of the tube and the expanded snare loop is perpendicular to the axis of the internal cavity or esophagus ready to capture the bolus, undesirable growths or objects.

The present invention is also directed towards a method of using the endoscopic snare net retrieval device for capturing and retrieving a bolus or a foreign body from an internal cavity of a patient, comprising: 1) using the driving member at the proximal end of the tube to fully retrieve the snare loop into the lumen of the tube at the distal end of the tube; 2) guiding the endoscopic snare net retrieval device into the interior of the internal cavity or the esophagus of a patient, encountering the bolus or objects to be removed; 3) actuating the drive member to release and to expand the snare loop at the distal end of the tube, such that the external suture or string is fully linear away from the tube and that the expanded snare loop is perpendicular to the axis of the internal cavity or the esophagus; 4) using the drive member to adjust the diameter or the enclosure of the expanded snare loop, and orienting the expanding snare loop relative to the axis of the internal cavity or the esophagus to capture the bolus or objects to be removed; and 5) removing the bolus, undesirable objects from the internal cavity or the esophagus using the snare loop, and exiting the endoscopic snare net retrieval device from the internal cavity or the esophagus.

The present invention is further directed towards the method of producing the endoscopic snare net retrieval device for capturing and retrieving a bolus or a foreign body from an internal cavity of a patient, comprising 1) fabricating a snare loop from a biocompatible metal and attaching it to a distal end of a control wire or cable for maneuverability; 2) securely coupling a resilient and flexible mesh to cover the area within the snare loop to ensure the effective capture of foreign bodies; 3) attaching a first end of a string or suture to the distal end of the control wire or cable, and attaching a second end of the string or suture to a point of the snare loop that is about 90° away from the distal end of the tube; 4) integrating the proximal end of the control wire or cable at the distal end of a tube into the the lumen of the tube, such that the proximal end of the wire reaches the proximal end of the tube, that the distal end of the control wire or cable is at or within the distal end of the tube and optionally the frist end of the suture or string passes through the outer wall at the distal end of the tube; 5) attaching a drive member to the proximal end of the control wire or cable to actuate the control wire or cable to fully retrieve the loop and suture into the tube, and/or to release or expand the loop at the distal end of the tube, to ensure the snare loop's perpendicular deployment when actuated by the drive member. This method emphasizes precision engineering to ensure that the device operates flawlessly within the confines of an internal cavity, providing a safe and reliable means for the retrieval of foreign bodies and growth tissues.

### BRIEF DESCRIPTION OF THE DRAWINGS

FIG. 1 depicts prior art endoscopic retrieval device utilizing “Roth Net”™.

FIG. 2 depicts an endoscopic snare net retrieval device in an expanded state and is fully supported by an external suture.

FIG. 3 depicts an endoscopic snare net retrieval device in an expanded state without the mesh and is fully support by an external suture.

### DETAILED DESCRIPTION OF THE INVENTION

An endoscopic snare net retrieval device for capturing and removing bolus, foreign bodies, or undesired growth tissues from an internal cavity of a patient, comprising: a snare loop; a mesh securely coupled to and cover the entirety of the area within the snare loop; a tube having a lumen configured and arranged therein; a control wire or cable that is disposed within a lumen of the tube such that the snare loop contains the mesh thereon is located at the distal end of the tube and is attached to a distal end of the control wire or cable; an external string or suture that has a first end coupled to a distal end of a control wire or cable via the outer wall of a distal end of the tube, and a second end that anchors the snare loop at a point on the snare loop that is outermost away from the distal end of the tube, and a drive member coupled to a proximal end of the control wire or cable and is exposed at a proximal end of the tube such that the actuation of the drive member move the snare loop via the control wire or cable, to fully retrieve the snare loop within the lumen of the tube at the distal end of the tube, or to release and expand the loop, the external string or suture, and the mesh coupled thereon at the distal end of the tube away from the tube, such that the expanded snare loop is about 90° apart from the distal end of the tube and the expanded snare loop is perpendicular to the axis of the internal cavity or esophagus ready to capture the bolus, undesirable growths or objects.

The expanded snare loop is preferably in a at least partially folding loop configuration that is center folded at an angle between 30° to 90° formed by the anchoring of the second end of the external string or suture, which is partially or fully expanded away from the distal end of the tube, such that the loop and the mesh is positioned by the external string or suture to have a configuration similar to a clam shell or a folded surface, to enclose or encircle the bolus, undesirable growths or objects, prior to adjusting the enclosure of the snare loop via the driving member to capture and remove the bolus, undesirable growths or objects from the internal cavity or the esophagus. The expanded snare loop could be partially or fully expanded, having a diameter of between 0.25 cm to 6 cm, preferably 0.5 cm to 5 cm, and more preferably 0.5 cm to 4.5 cm.

The endoscopic snare net retrieval device is essentially a perpendicularly deploying snare net. The fully deployed snare net is perpendicular to the axis of the internal cavity or the esophagus, thereby obviating the need to “blindly and forcibly” slide a snare net alongside a very tightly wedged food bolus. The endoscopic snare net retrieval device will not only decrease the risk of perforation, but it will cut down on the time needed to clear the esophagus of obstructions. Further, the endoscopic snare net retrieval device can pass through the biopsy channel and does not require additional modifications to endoscopes.

The present invention is also directed towards a method of using the endoscopic snare net retrieval device for capturing and retrieving a bolus or a foreign body from an internal cavity of a patient, comprising: 1) using the driving member at the proximal end of the tube to fully retrieve the snare loop into the lumen of the tube at the distal end of the tube; 2) guiding the endoscopic snare net retrieval device into the interior of the internal cavity or the esophagus of a patient, encountering the bolus or objects to be removed; 3) actuating the drive member to release and to expand the snare loop at the distal end of the tube, such that the external suture or string is fully linear, away from the tube and that the expanded snare loop is perpendicular to the axis of the internal cavity or the esophagus; 4) capturing by adjusting the diameter or the enclosure of the expanded snare loop, and orienting the expanding snare loop relative to the axis of the internal cavity or the esophagus; 5) removing the bolus, undesirable objects from the internal cavity or the esophagus using the snare loop; 6) wherein upon removing, the drive member is manipulated to adjust the diameter of the loop so as to facilitate existing of said endoscopic snare net retrieval device from said internal cavity of said patient; and 7) exiting the endoscopic snare net retrieval device from the internal cavity or the esophagus.

The manufacturing process of the endoscopic snare net retrieval device comprising a first step to producing a snare loop formed from a high-grade, biocompatible metal such as stainless steel or titanium. This metal molded and tempered to achieve an optimal balance of flexibility and rigidity, ensuring that the snare loop can navigate the endoscopic pathways while retaining its shape for effective capture.

Secondly a mesh is is cut and shaped, then bonded or jointed to the snare loop and affixed within the snare loop's perimeter using specialized biocompatible adhesives or by ultrasonic welding, in order to create a seamless and secure bond that withstands the mechanical stresses of operation. Suitable mesh material is selected from the group consisting of polypropylene, polyester, ePTFE and mixtures thereof.

After the mesh has been incorporated onto the snare's loop, attaching a first end of a string or suture to the distal end of the control wire or cable, and attaching a second end of the string or suture to a point of the snare loop that is outermost away from the distal end of the tube, such that this point is about 90° away from the distal end of the tube. Then, integrating the proximal end of the control wire or cable at the distal end of a tube into the lumen of the tube, such that the proximal end of the wire reaches the proximal end of the tube, that the distal end of the control wire or cable is at or within the distal end of the tube, and that optionally the first end of the suture or string passes through or located by the outer wall at the distal end of the tube.

Preferably, the snare loop is configured as a at least partially folding loop having a center folded of between 30° to 90° at the point connecting to the second end of the external string or suture that is fully expanded away from the distal end of the tube, such that the loop and the mesh is positioned by the external string or suture to have a configuration similar to a clam shell or a folded surface. The lumen of the tube has the dimensions to accommodate the control wire, allowing smooth and responsive actuation of the snare loop. The distal end of the tube allows a snug fit for the external suture or string such that upon deployment, the snare loop expands perpendicularly to the axis of the internal cavity or esophagus, thus avoiding the need for forceful insertion past tightly lodged boluses.

Finally, attaching a drive member to the proximal end of the control wire or cable to actuate the control wire or cable to fully retrieve the loop and suture into the tube, and/or to release or expand the loop at the distal end of the tube, to ensure the snare loop's perpendicular deployment when actuated by the drive member. Quality control checks are performed throughout the manufacturing process to validate the function of the actuation mechanism, the integrity of the snare loop and mesh, and the overall usability of the device. This comprehensive manufacturing methodology ensures that the endoscopic snare net retrieval device is reliable, safe, and ready for medical application.

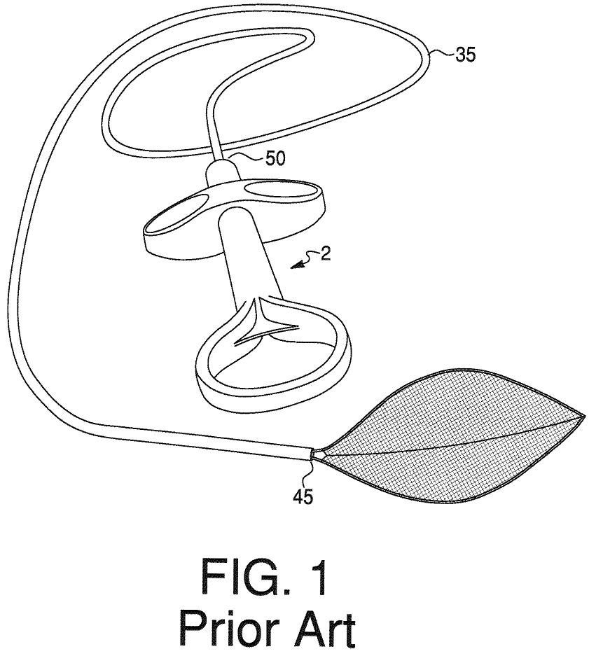

As shown in FIG. 1, “Roth Net”™ is general known as a retrieval device within the lumen of a tube, and its inflexibility of such device to pass between bolus and esophageal wall.

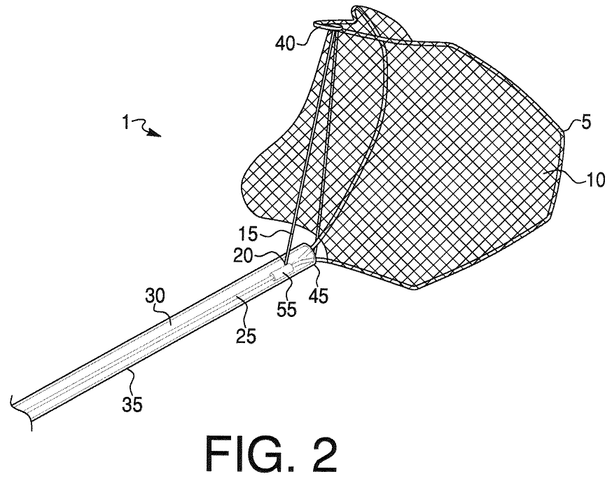

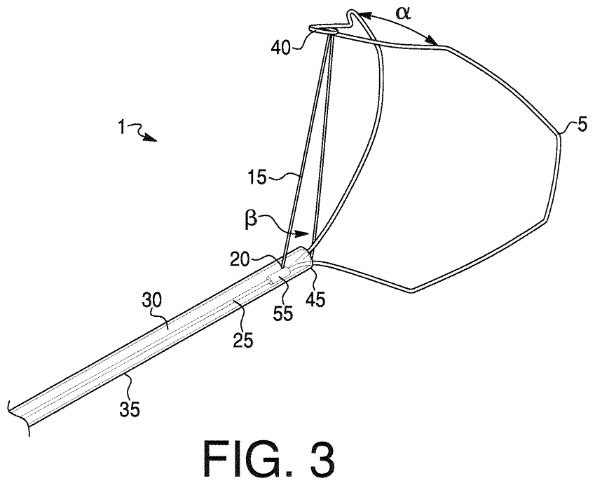

In the present invention, as shown in FIGS. 2 and 3, endoscopic snare net retrieval device 1 has a snare loop 5 and mesh 10 coupled thereon to cover the entire area of snare loop 5, such that snare loop 5 is securely attached to cable 25 via connection means 55 and is fully retrievable into lumen 30 of tube 35 at distal end 45. Snare loop 5 is configured as a at least partially folding snare loop 5 having a center folded angle α at between 30° to 90° away from an external string or suture 15 that is fully expanded away from the distal end 45 of the tube, such that snare loop 15 and mesh 10 is positioned by external string or suture 15 to have a configuration similar to a clam shell or a folded surface. In an expanded state, a first end of external suture or string 15 is attached to cable 25 at connection means 55 in tube 35 via opening 20 on the outer wall of tube 35, and a second end of external suture or string 15 anchors snare loop 5 at anchor point 40, such that the external suture or string 15 restricts and tightens snare loop 5 to be perpendicular with tube 35 and/or esophagus (not shown), and that anchor point 40 is at an angle β 90° away from the distal end 45 The retrieval and expansion of the cable 25 is aided by a drive member (not show) that is similar to drive member 2 of FIG. 1, and located on proximal end 50 of tube 35 of FIG. 1.

FIG. 3 shows endoscopic snare net retrieval device 1 without mesh 10, to better illustrates the perpendicular nature of loop 5 in an expanded state.

1. An endoscopic snare net retrieval device comprising: a snare loop; a mesh securely coupled to and cover the entirety of the area within said snare loop; a tube having a lumen configured and arranged therein; a control wire or cable that is disposed within said lumen of said tube, such that said snare loop contains said mesh thereon is located at said distal end of said tube and is attached to said distal end of the control wire or cable; an external string or suture that has a first end coupled to a distal end of said control wire or cable, and a second end anchors said snare loop away from said distal end of said tube; and a drive member coupled to a proximal end of said control wire or cable and is exposed at a proximal end of the tube, such that an actuation of said drive member moves said snare loop via said control wire or cable, to fully retrieve said snare loop within said lumen of the tube at said distal end of said tube, or to release and to expand said snare loop, said external string or suture and said mesh coupled thereon at said distal end of said tube away from said tube such that said snare loop is about 90° apart from said distal end of said tube.

2. The endoscopic snare net retrieval device of claim 1, wherein said first end of said external string or suture is coupled to a distal end of said control wire or cable via the outer wall of a distal end of said tube.

3. The endoscopic snare net retrieval device of claim 1, wherein said second end of said external string or suture anchors said snare loop at the outermost point away from said distal end of said tube.

4. The endoscopic snare net retrieval device of claim 3, wherein the anchoring of said snare loop by said second end of said external string or suture allows said snare loop to configured as a at least partially folding loop having a center folded having an angle of between 30° to 90° formed by said anchoring.

5. The endoscopic snare net retrieval device of claim 4, wherein said external string or suture is partially or fully extended away from said distal end of said tube.

6. The endoscopic snare net retrieval device of claim 4, such that in said expanded state, said loop and said mesh are positioned by said external string or suture to have a configuration similar to a clam shell or an enclosure to capture a bolus, undesirable growth, or foreign object.

7. The endoscopic snare net retrieval device of claim 1, wherein said expanded snare loop is perpendicular to the axis of an internal cavity or esophagus of a patient ready to capture bolus, undesirable growths, or objects.

8. The endoscopic snare net retrieval device of claim 1, wherein said expanded snare loop has a diameter of between 0.5 cm to 5 cm.

9. The endoscopic snare net retrieval device of claim 1, wherein said snare loop formed from a high-grade, biocompatible metal such as stainless steel or titanium.

10. The endoscopic snare net retrieval device of claim 1, wherein mesh material is selected from the group consisting of polypropylene, polyester, ePTFE and mixtures thereof.

11. A method of using the endoscopic snare net retrieval device of claim 1 for capturing and retrieving a bolus or a foreign body from an internal cavity of a patient, comprising: 1) using said driving member at said proximal end of said tube to fully retrieve said snare loop including said mesh thereon into said lumen of said tube at said distal end of said tube; 2) guiding said endoscopic snare net retrieval device into the interior of an internal cavity or the esophagus of a patient, encountering bolus or objects to be removed; 3) actuating said drive member to released or expand said snare loop at said distal end of said tube, such that said external suture or string is fully linear away from said tube and that said expanded snare loop is perpendicular to the axis of said internal cavity or said esophagus; 4) capturing said bolus or objects to be removed by adjusting said diameter or said enclosure of said expanded snare loop, and orienting said expanding snare loop relative to said axis of said internal cavity or said esophagus; and 5) removing said bolus, undesirable objects from said internal cavity or said esophagus using said snare loop, and exiting said endoscopic snare net retrieval device from said internal cavity or said esophagus of said patient.

12. The method of using the endoscopic snare net retrieval device of claim 11 wherein said step 3) further orienting the expanded snare loop relative to the axis of the internal cavity to facilitate capture of the bolus or foreign body.

13. The method of using the endoscopic snare net retrieval device of claim 11, wherein said step 3) further comprising anchoring of said snare loop by said second end of said external string or suture to form said snare loop as a at least partially folding loop in clamshell-like configuration having a center folded having an angle of between 30° to 90°.

14. The method of using the endoscopic snare net retrieval device of claim 11, wherein upon capturing, said drive member is manipulated to adjust said diameter of said snare loop so as to facilitate existing of said endoscopic snare net retrieval device from said internal cavity of said patient.

15. A method of producing an endoscopic snare net retrieval device, comprising 1) fabricating a snare loop from a biocompatible metal and attaching it to a distal end of a control wire or cable; 2) securely coupling a resilient and flexible mesh to cover the area within said snare loop; 3) attaching a first end of a string or suture to said distal end of said control wire or cable, and attaching a second end of said string or suture to a point of said snare loop such that said snare loop is about 90° apart from said distal end of said tube; 4) integrating said proximal end of said control wire or cable at said distal end of a tube into the lumen of said tube, such that said proximal end of said wire reaches said proximal end of said tube, and that said distal end of said control wire or cable is at or within said distal end of said tube and; 5) attaching a drive member to said proximal end of said control wire or cable to actuate said control wire or cable to fully retrieve said loop and suture into said tube, and/or to release or expand said loop at said distal end of said tube.

16. The method of claim 15, wherein at step 3) said first end of said suture or string passes through the outer wall at said distal end of the tube to attach to said distal end of said control wire or cable.

17. The method of claim 15, wherein at step 3) said point that said second end of said external string or suture anchors said snare loop is the outermost point on said snare loop away from said distal end of said tube.

18. The method of claim 17, wherein at step 3) wherein the anchoring of said snare loop by said second end of said external string or suture allows said snare loop to configured as a at least partially folding loop having a center folded having an angle of between 30° to 90° formed by said anchoring.

19. The method of claim 15, wherein at step 5) when said snare loop is expanded, said snare loop is perpendicular to the axis of an internal cavity or esophagus of a patient ready to capture bolus, undesirable growths, or objects.

20. The method of claim 15, wherein in said expanded state, said loop and said mesh are positioned by said external string or suture to have a configuration similar to a clam shell or an enclosure to capture a bolus, undesirable growth, or foreign object.

21. The method of claim 15, wherein said expanded snare loop has a diameter of between 0.5 cm to 5 cm.

22. The method of claim 15, wherein said snare loop formed from a high-grade, biocompatible metal such as stainless steel or titanium.

23. The method of claim 15, wherein mesh material is selected from the group consisting of polypropylene, polyester, ePTFE and mixtures thereof.
